(19) United States
(12) Patent Application Publication
(10) Pub. No.: US 2007/0172803 A1
(43) Pub. Date: Jul. 26, 2007

(54) SKILL EVALUATION

(76) Inventors: Blake Hannaford, Seattle, WA (US); Jacob Rosen, Seattle, WA (US); Jeffrey D. Brown, Warsaw, IN (US); Timothy Mariusz Kowalewski, Tacoma, WA (US); Mika Sinanan, Brier, WA (US); Lily Chang, Boston, MA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/466,269

(22) Filed: Aug. 22, 2006

Related U.S. Application Data
(60) Provisional application No. 60/711,514, filed on Aug. 26, 2005.

(51) Int. Cl. G09B 23/28; A61B 17/00; G06G 7/48

(52) U.S. Cl. 434/262; 606/1; 703/11

(57) ABSTRACT

Software tools, methods and apparatus for objectively assessing surgical and medical procedural skills are described. Data corresponding to performance of a manipulative task by a subject is modeled using Markov modeling techniques and compared with stored models corresponding to each of a plurality of proficiency levels. A particular proficiency level is selected based on proximity of the subject data relative to each of the stored models.

### CROSS-REFERENCE TO RELATED PATENT DOCUMENTS

This patent document claims the benefit of priority, under 35 U.S.C. Section 119(e), to Blake Hannaford, U.S. Provisional Patent Application Ser. No. 60/711,514, entitled “SKILL EVALUATION,” filed on Aug. 26, 2005 (Attorney Docket No. 2082.006PRV).

### STATEMENT OF GOVERNMENT RIGHTS

The invention was made with Government support under contract or grant number DAMD17-97-1-7256 entitled “Force/Torque Signatures in Minimally Invasive Surgery: Quantification of Skill and Improvement of Outcomes,” project period 6/1997-5/1999, awarded by the Defense Advanced Research Projects Agency (DARPA) and under contract or grant number W81XWH-04-1-0464 entitled “Markov Models,” project period March 2004-May 2006, awarded by the Department of Defense (DOD). An Information Technology Research (ITR) award from the National Science Foundation (NSF) via the John Hopkins University supported this work. The Government has certain rights in this invention.

### TECHNICAL FIELD

This document pertains generally to evaluations, and more particularly, but not by way of limitation, to skill evaluation.

### BACKGROUND

Human performance of a task, such as surgery, is evaluated for various reasons, including for example, developing skills and identifying expertise. Objective and subjective evaluation criteria can be established for evaluating or judging the performance of a subject. Some examples of tasks in which a subject uses physical controls to manipulate a mechanism include surgery, driving a vehicle and operating machinery.

Typical methods of evaluating performance entail human oversight and are, thus, financially burdensome and often imprecise.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 includes a diagram showing selected modalities for surgery.

FIG. 2 includes a table of definitions for 15 states based on a spherical coordinate system.

FIG. 3 illustrates time charts for left and right endoscopic tools of a surgical robot system during a surgical procedure.

FIG. 4 illustrates vector representation of exemplary data.

FIG. 5 illustrates an exemplary cluster center.

FIG. 6 illustrates selected degrees of freedom.

FIGS. 7A and 7B illustrate a finite state diagram.

FIG. 8 illustrates exemplary Markov models represented as coded probabilistic maps.

FIG. 9 schematically illustrates statistical distances relative to an expert.

FIG. 10 illustrates normalized Markov model-based statistical distances.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

Overview

The present subject matter includes methods and systems for evaluating skills. Exemplary methods utilize a Markov model or Hidden Markov model for analyzing the departure of a specific signal from what is expected by that model.

The present subject matter is described in this document largely based on Markov and hidden Markov models. Nevertheless, other types of models are also contemplated, including algorithmic or rule-based models, dynamical system models and statistical models (of which Markov and hidden Markov models are but two examples).

In one example, the performances of surgical skills on a pig by several participants were recorded and a model based on data generated from experts performing the skills has been created. The present subject matter distinguishes between signals generated by experts and non-experts and can be applied to non-surgical manipulative tasks including, human or non-human operation of a machine. For example, the present subject matter can facilitate analysis of manipulations of physical controls used to operate a mechanism, such as driving a vehicle (steering wheel and pedals), flying an aircraft (yoke and pedals), operating machinery (such as a crane) and minimally invasive surgery.

Markov and hidden Markov models are exemplary statistical models which can be used for voice recognition of speech. Models of speech sounds are created in a controlled manner and a sample sound is recognized based on a comparison of the sample sound with those models. Statistical models, such as Markov and hidden Markov models, can tolerate variations in utterance of a particular word.

In the present subject matter, electrical signals derived from surgical instruments are used as a source input. The electrical signals are generated by sensors coupled to a surgical instrument when manipulated by operators performing at various skill levels. Surgical skill models are developed based on the recorded information. Once trained, data recorded by other surgeons (including experts and novices) are examined using the model. The model can be used to identify expert surgeons in a group. In one example, the present subject matter includes a skill measurement tool.

The analysis of the data recorded during surgery can be done off-line. That is, data analysis (and expert identification) is conducted after completion of the surgical procedure.

In one example, the data analysis is conducted in real time. That is, data processing and quantification of the skill level of subjects is performed concurrent with data acquisition.

In one example, large amounts of recorded data is compressed and simplified using vector quantization. Vector quantization was initially developed for image compression and it is adapted for use in the present subject matter.

The method includes receiving electric signals associated with a subject performing a particular task. Greater number of signals provides improved performance. In one example, the method includes receiving data recorded by experts to train a model.

In one example, a surgical robot is used to train subjects and subject performance evaluation is generated in real time. Feedback provided by the present system can augment skill development and reduce the burden of supervision.

In one example, a robotically controlled interface is coupled to one or more simulators for training purposes.

In one example, subjects are scored on their performance based on a simulated or actual manipulative task. In one example, performance is evaluated using a simulation prior to performing an actual complex procedure. Feedback derived from the evaluated simulation can be used to tailor actual performance. For example, surgeon performance using a surgical simulator can be evaluated prior to conducting actual surgery on a patient. The evaluation may reveal that the subject's performance is inferior to that of an expert because of fatigue or other correctable factor.

In one example, an interface includes a layer operating in the background of the surgical environment (actual, virtual or robotically controlled) which can interject upon detection of a departure from an expert performance. For example, if the conduct of a lower skilled surgeon is detected, then at a critical procedure, the layer will interrupt and prevent harmful movement or interrupt and suggest an improved course or provide tactile feedback (haptic) sensations to cause the surgeon to alter their performance. The layer can be implemented in hardware or in instructions executed by a computer of the present subject matter. In one example, the background layer fulfills a supervisory role as to a manipulative task.

The Markov decision process makes decisions by prioritizing possible choice as measured by evolving values criteria.

Assessing Skill with Medical Simulators

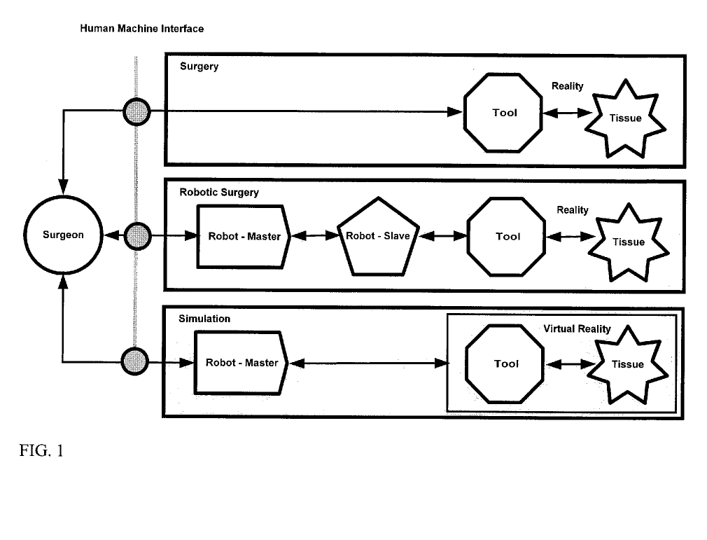

In the surgical context, procedurally-oriented skills can be performed utilizing three different modalities, (a) during actual open or minimally invasive clinical procedures; (b) in physical or virtual reality simulators with or without haptic feedback; and (c) during interaction with surgical robotic systems, as shown in FIG. 1. During open or minimally invasive surgical (MIS) procedures, the surgeon interacts with the patient's tissue either directly with his/her hands or through the mediations of tools. Surgical robotics enables the surgeon to operate in a tele-operation mode with or without force feedback using a master/slave system configuration. In this mode of operation, visualization is obtained from either an external camera or an endoscopic camera. Incorporating force feedback, allows the surgeon to feel through the master console the forces being applied on the tissue by the surgical robot, the slave, as he/she interacts with it from the master console. For training in a simulated virtual environment, the surgical tools, the robot-slave, and the anatomical structures are replaced with virtual counterparts. The surgeon interacts with specially-designed input devices, haptic devices when force feedback is incorporated, that emulate surgical tools, or with the master console of the robotic system itself, and perform surgical procedures in virtual reality.

In each modality, the surgeon is separated from the treated tissue or medium by an instrument or a mechanical interface. In some examples, the interface includes a virtual component. The intermediate modality in all these examples can be considered interchangeable. A common element of these modalities is the human-machine interface in which visual, kinematics, dynamic, and haptic information is shared between the surgeon and the various modalities. This interface can provide multi-dimensional data to objectively assess technical surgical skill within the general framework of surgical ability.

The algorithm used for objective assessment of skill is independent of the modality actually used and therefore, the same algorithms can be incorporated into any of these technologies. Objective methodologies for assessing task or skill competence and performance can be used to enhance training, reduce cost and improve competency.

In one example, the surgical task is deconstructed or decomposed to expose and analyze the internal hierarchy of tasks. Task decomposition is associated with defining selected elements of the manipulative process. For example, in surgery, the procedure is divided into steps, stages, or phases with defined intermediate goals. Additional hierarchical decomposition is based on identifying tasks or subtasks and actions or states. Low-level elements of the task decomposition are associated with quantify measurable parameters. Definition of these states along with measurable, quantitative data allows for modeling of surgical tasks or medical examination.

The present subject matter can be applied to the various modalities and includes decomposing the medical procedure (such as an examination or surgical task) into fundamental states associated with discrete observations. The task is represented by a statistical model such as a multi-state Markov model, a hidden Markov model or other such model. A performance of a test subject is evaluated based on the statistical distance calculated between the test subject and at least one stored model. In one example, the stored models correspond to performance of the task at various skill levels, including that of a novice and an expert. The analysis can be conducted in real-time and provide feedback during the performance. Feedback, in various examples, can be in the form of audio, visual or tactile. The present subject matter can be used with various modalities and systems (including robotic systems and simulators) for evaluating performance of a manipulative task.

In the present subject matter, a prime element is modeled by a finite state. In the context of Markov modeling and speech recognition, the prime element is the spoken word. The prime element in the surgical context relates to tool-tissue interaction or hand-tissue interaction. Within a particular tool-tissue interaction or hand-tissue interaction, variations in forces and torque magnitudes can be noted for different skill levels and, in the context of speech recognition, this relates to variations in word pronunciation. The various force and torque magnitudes are simulated by discrete observations in the model. A sequence of tool-tissue or hand-tissue interactions comprise the steps of a medical procedure having intermediate and specific outcomes, and by analogy in the speech recognition context, a sequence of words represent a sentence or chapter.

A variety of sensors are used to generate signals corresponding to, for example, completion time, work space, force, position and tool path.

### EXAMPLE

In one example, a physical simulator in the form of an instrumented teaching-mannequin representing the female pelvis and the breast exam, male prostate exam, and endotracheal intubation was used. Data was acquired from approximately 1800 students and clinicians, including quantitative measures of hands-on clinical exam techniques used while performing procedures. Background information for the students and clinicians, and a database of outcome measures including the user's clinical assessment scores and independent skilled observer ratings of the users' techniques while performing these examinations or procedures in physical simulators, was also collected.

Sensors coupled to surgical robotic systems were used to collect data on surgical tool positions and the torque commands between the master unit and the robotic instrument actuators.

Markov modeling, according to the present subject matter, provides an objective assessment of medical/surgical skills in a manner transparent to modality.

In one example, data mining is performed on a database corresponding to a manipulative task. A surgical robot provides data generated by sensors while performing surgical tasks on animal and human subjects.

In one example, two-handed, instrumented endoscopic tools and Markov models are used to perform task decomposition and objective skill assessment with the Markov modeling approach. Sensor arrays coupled to the tools and robotic systems provide quantitative data to allow data mining and clustering and multi-state Markov modeling and analysis of the particular tasks.

Objective assessment of surgical competence during minimally invasive surgery procedures is a multi-dimensional problem. Minimally invasive surgery (MIS) refers to a surgical procedure involving a minimally invasive surgical setup. Physiological constraints (stress, fatigue), equipment constraints (camera rotation and port location), team constraints (nurses), and physician ability are representative parameters that affect the outcome of a MIS procedure. Ability, with respect to surgery, is defined as the natural state or condition of being capable; innate aptitude (prior to training), which an individual brings for performing a surgical task. Minimally invasive surgery ability includes cognitive factors (knowledge and judgment) and technical factors (psychomotor ability, visio-spatial ability and perceptual ability). By definition, fundamental psychometric abilities are fixed at birth or early childhood and show little or no learning effect. However training enables the subject to perform as close as possible to his or her inherent psychometric abilities.

The methodology for objectively assessing surgical skill (as a subset of surgical ability), according to the present subject matter, includes objective and quantitative analysis. Such methodology is enabled by using instrumented tools, measurements of the surgeon's arm kinematics, gaze patterns, physical simulators, a variety of virtual reality simulators (those with and without haptics) and robotic systems. An instrumented tool can be used to generate data corresponding to kinematics (position, velocity, acceleration, and jerk), dynamics (force, and torque), contact information between the tool and the medium (a.go. real tissue or simulated tissue) and recorded display of the scene in the proximity of the tool.

Regardless of the modality being used or the clinical procedure being studied, task deconstruction or decomposition is one component of an objective skills-assessment methodology. Exposing and analyzing the internal hierarchy of tasks provides an objective means for quantifying training and skills acquisition.

Task decomposition is associated with defining the prime elements of the manipulative task. In surgery, a particular procedure is divided into steps, stages, or phases with well-defined intermediate goals. Additional hierarchical decomposition is based upon identifying tasks or subtasks including a sequence of actions or states. In addition, other measurable parameters such as workspace completion time, tool position, and forces and torques can be analyzed. Selecting low-level elements of the task decomposition allows one to associate these elements with quantifiable and measurable parameters. The definition of these states, along with measurable, quantitative data, are used for modeling and examining surgical tasks as a process.

In the proposed study, an analogy between minimally invasive surgery (MIS) and the human language inspires the decomposition of a surgical task into its prime elements. Modeling the sequential element expressions using a multi-finite states model (for example, a Markov model) reveals the internal structure of the surgical task which is utilized in assessing surgical performance. Markov modeling (MM) and hidden Markov modeling (HMM), a subset of MM, are used to characterize manipulative tasks.

Within the context of the three modalities (direct surgery/clinical examination, simulated procedures—either physical or virtual, and surgical robot), the procedure can be summarized as follows:
a) decompose the clinical task into fundamental states associated with discrete events (observations);
b) represent the task using a statistical model such as a multi-state Markov model; and
c) determine statistical distances between a subject performance and models representing subjects with various skill levels.

In one example, the present subject matter includes procedures for analyzing a database acquired from two modalities (simulator and instrumented surgical tools) using vector quantization algorithms.

According to one example, a method includes decomposing the task using expert knowledge and developing the Markov model architectures, training the Markov models based on the processed data, developing the learning curves based on measuring the statistical similarity between the models representing subjects at different levels of surgical training to enable an objective assessment of surgical skills and generalizing the methodology for assessing skill in the three modalities.

In the context of battlefield conditions, for example, military medical personnel may be called upon to perform tasks that may exceed the complexity or skill of a civilian medical personnel. Even extended experience in a civilian trauma center may be inadequate to prepare military personnel to perform under realistic conditions. As such, simulators are valuable tools in training military personnel. In addition, a mechanism for assessing skill can be helpful in a simulator and in particular, a simulator used to train military medical care providers.

Among other applications, a statistical model, such as a Markov model, can provide a tool in developing a methodology for studying models of the human operator in complex interactive tasks with machines.

Databases and Data Collection

A particular surgical robot, known popularly as the BlueDRAGON, is a system developed at the University of Washington for acquiring the kinematics and the dynamics of two endoscopic tools along with the visual view of the surgical scene while performing a MIS procedure. The system includes two four-bar passive mechanisms attached to two endoscopic tools. During a minimally invasive surgical procedure, the endoscopic tool is inserted into the body through a port located, for example, in the abdominal wall. The tool is rotated around a pivot point within the port that is generally inaccessible for sensors aimed to measure rotation of the tool. The position and orientation of the tool, with respect to the port, is tracked by sensors that are incorporated into the joints of the mechanism. The two mechanisms are equipped with three classes of sensors.

A first class of sensor include position sensors (such as potentiometers) incorporated into four of the joints of the mechanisms for measuring the position, orientation and translation of the two instrumented endoscopic tools attached thereto. In addition, two linear potentiometers are attached to the handles of the tools and used for measuring the endoscopic handle and tool tip angles.

A second class of sensors include three-axis force/torque (F/T) sensors (with holes drilled at their center) that are inserted and clamped to the proximal end of the shafts of the endoscopic tools. In addition, double beam force sensors are inserted into the handles of the tools for measuring the grasping forces at the hand-tool interface.

A third class of sensors include contact sensors, based on a resistance-capacitance (RC) circuit, which provides a binary indication of tool-tip/tissue contact.

Data measured by the sensors are acquired using two 12-bit USB A/D cards sampling the 26 channels (4 rotations, 1 translation, 1 tissue contact, and 7 channels of forces and torques from each instrumented grasper) at a frequency of 30 Hz. In addition to data acquisition, the synchronized view of the surgical scene is incorporated into a graphical user interface displaying data in real-time.

Preliminary tests acquiring data at a sampling rate of 1 KHz indicated that 95% of the signals accumulated energy is in a bandwidth 0-5 Hz. In addition, a graphical user interface (GUI) is provided to display information measured by the surgical robot in real-time while incorporating endoscopic view of the surgical scene acquired by the endoscopes video camera. On the top right side of the GUI, a virtual representation of the two endoscopic tools are shown along with vectors representing the instantaneous velocities. On the bottom left a three dimensional representation of the forces and torque vectors are presented. Surrounding the endoscopic image are bars representing the grasping/spreading forces applied on the handle and transmitted to the tool tip via the tool's internal mechanism, along with virtual binary LED indicating contact between the tool tips and the tissues.

A representative physical simulator is popularly known as the E-Pelvis. The E-pelvis is a physical simulator developed at Stanford University that consists of a partial mannequin (umbilicus to mid-thigh) constructed in the likeness of an adult human female. The mannequin is instrumented internally with force sensors that are connected to a computer having a graphical user interface for providing a real-time visual feedback. Test subjects perform simulated clinical female pelvic examinations on the mannequin and the data is collected at a sampling frequency of 30 Hz and stored in memory for off-line analysis.

A representative surgical robot system, popularly known as DaVinci, is commercially available from Intuitive Surgical (Sunnyvale, Calif.) and is FDA approved for selected surgical procedures. The system is equipped with an interface card that allows passive acquisition of internal variables of the robot during operation. Examples of data generated include position of the surgical tools and motor commands. The data is sampled at 30 Hz, displayed in real time by using a user interface and stored for off-line analysis.

Protocol for the Surgical Robot

The protocol using the surgical robot included collecting data from task performances conducted by surgeons having different levels of expertise. In one example, the performances of 30 surgeons were monitored. Levels of expertise ranged from surgeons in training to surgical attending physicians. Five subjects in each group represented the five years of surgical training, (5×R1, R2, R3, R4, R5-where the numeral denotes year of training) and five expert surgeons. For the purpose of this example, an expert surgeon (E) was defined as a board certified laparoscopic surgeon who performed at least 800 surgeries and practices medicine as an attending physician. Each subject was given instruction through a multimedia presentation on how to perform three basic surgical tasks involving (1) tying an intracorporeal knot; (2) manipulating tissue; and (3) tissue dissection. The multimedia presentation included a written description of the task and a video clip of the surgical scene with audio explanation of the task. Subjects were then given 15 minutes in which to complete this task in a swine model.

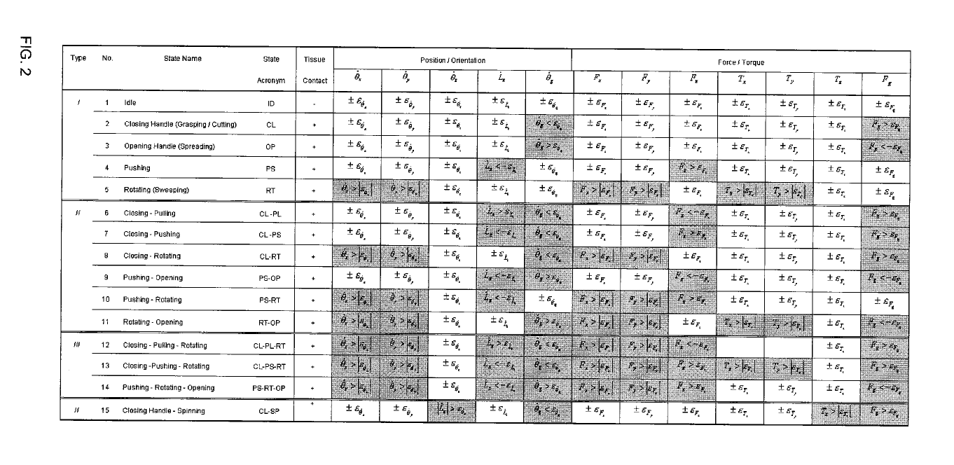

In addition to the surgical task, each subject performed 15 predefined tool/tissue and tool/needle-suture interactions as shown in FIG. 2. The definitions of the 15 states are based on a spherical coordinate system with an origin at the port. Each state features a unique set of angular/linear velocities, forces and torques. A non-zero threshold value is defined for each parameter by ε. The states' definitions are independent from the tool tip being used. For example, the state defined as Closing Handle might be associated with grasping or cutting if a grasper or scissors are being used respectively.

The kinematics (that is, the position/orientation (P/O) of the tools in space with respect to the port), and the dynamics (that is, forces and torque—F/T—applied by the surgeons on the tools) of the left and right endoscopic tools along with the visual view of the surgical scene were acquired by a passive mechanism coupled to the surgical robot. This data provided the F/T and velocity signatures associated with each interaction that were then used as the model observations associated with each state of the model.

Protocol for the Physical Simulator

The experimental protocol for the simulator included 400 students and 375 clinicians performing pelvic examinations using the simulator. The data include forces as a function of time recorded from sensors distributed in the simulator. In addition, background information on all of the users was also recorded. These records include a database of outcome measures, the user's clinical assessment scores, and independent skilled observer ratings of the users' techniques while performing examinations or procedures on the simulators.

Data Analysis

The methodology for analyzing the data includes a multi-step processes of data reduction starting from multi-dimensional raw data and ending with a single objective performance score. The methodology is linked directly to the physics of the medium being treated. Data processing provides insights into the process being analyzed as opposed to a black box approach where only the inputs and outputs are well defined and the modal internal architecture is arbitrarily selected and unlinked to the physical world.

Multi-Dimensional Raw Data

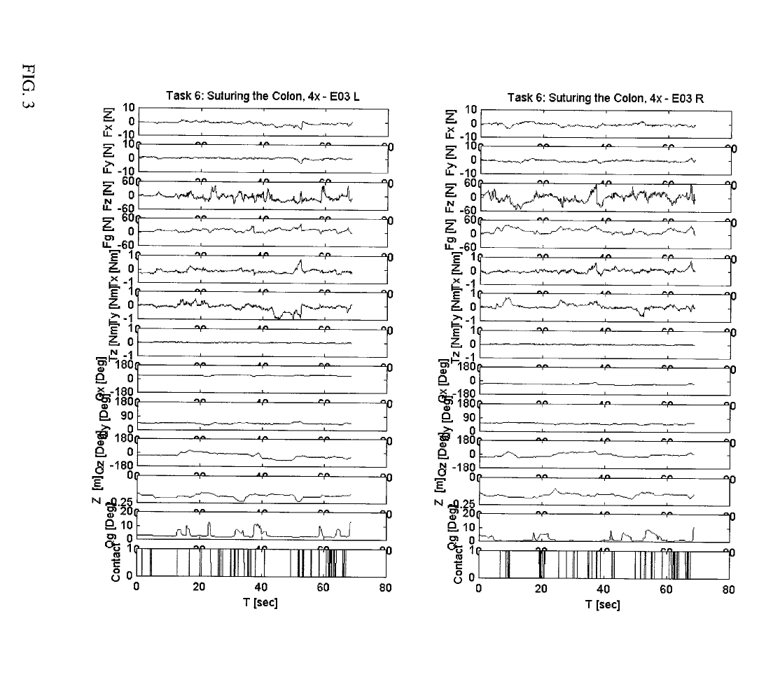

Multi-dimensional data was collected as a function of time for each modality under study. Time charts of the typical plots are depicted in FIG. 3. The exemplary data of FIG. 3 was acquired from the left and the right endoscopic tools of a surgical robot system during suturing of the colon by an expert surgeon in an MIS setup. Forces torques angles and contact information are plotted as a function of time.

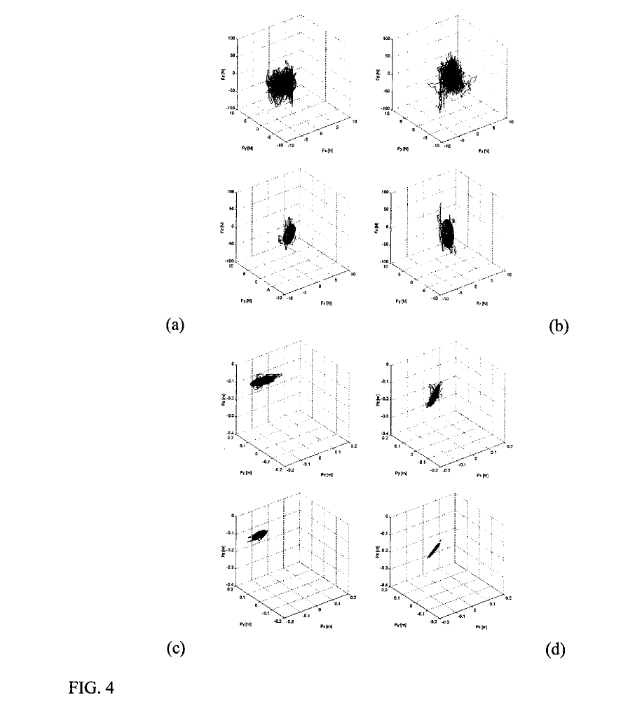

The vector representation of the data allows spatial graphical representation rather than time charts. Vector representation of exemplary data is shown in FIG. 4. The forces and torques (F/T) vectors are depicted as arrows with origins located at the port, and the lengths and orientations changing as a function of time based on the F/T applied by the surgeon's hand on the tool while interacting with the tissues, needle and suture. In a similar fashion, the traces of the tool tips with respect to the ports can be plotted as their positions changed during the surgical procedure using a spatial graphical form. Typical raw data of F/T and tool tip position traces were plotted using three dimensional graphs for the left and right endoscopic tools as measured by the surgical robot while performing the MIS intracorporeal knot tie by junior trainee (denoted as model R1 and shown in FIGS. 4A and 4C) and expert surgeon (denoted as model E and shown in FIGS. 4B and 4D). Forces are shown in FIGS. 4A and 4B and tool tip position is shown in FIGS. 4D and 4C. The ellipsoids contain 95% of the data points.

The complexity of the surgical task and the multi-dimensional data can be noted in the raw data. This complexity can be resolved, in part, by decomposing the surgical task into primary elements, thus enabling insights into the clinical procedure as a process.

Vector Quantization

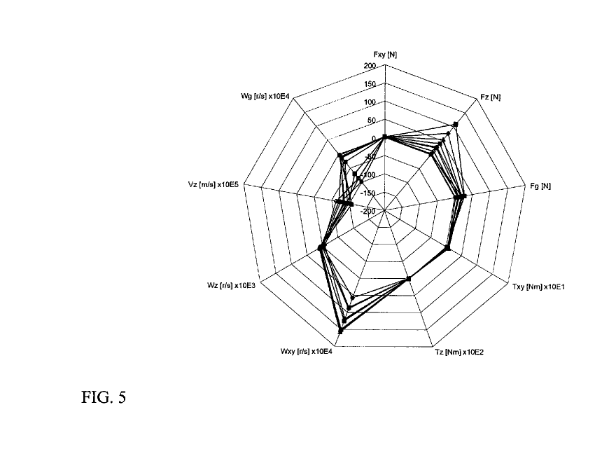

Data quantization is used to reduce the dimensions of the data. The data can be envisioned as a non-homogeneous discrete cloud encompassing the acquired data points, as illustrated in FIG. 5. As part of the iterative data quantization process, the vector quantization algorithm (e.g. K-means) searches for high-density regions in the non-homogeneous discrete cloud and assigns a cluster center to each one of the regions identified in the cloud. The number of clusters is bounded by the number of data points in the database (maximal value) and 1 (minimal value). In the extreme case where the number of clusters is equal to one, the cluster center vector represents the mean of that data. There are several techniques to define the optimal number of cluster centers in order to minimize the information that is lost due to data reduction associated with this process. Using the human language as an analogy, each data point associated with a specific cluster center represents a variant of a standard pronunciation defined by the cluster center.

Each cluster center can be defined by a discrete symbol (e.g. S̅₁, S̅₂, . . . S̅ₖ etc.) forming a codebook. The database is then encoded into this codebook. Each point in the database is associated with only one cluster center in the codebook in which the distance between the selected cluster center and the data point is minimal. After encoding, the database contains a list of symbols as a function of time. The encoding process generates a substantial reduction in the dimensionality of the database. Encoding also reduces the data from a multi-dimensional space (e.g. 12 dimensional space in case of the MIS database) to a single dimensional space of symbols (150 symbols in the case of MIS database) representing the closest cluster centers as a function of time.

In one example, the number of states of a Markov model is selected based on user-selected criteria. For example, a 30-state Markov model can be used to represent two tools working collaboratively or a 3-state or 15-state hidden Markov model can be used to represent a single tool.

Each one of the 15 states was associated with a unique set of forces, torques, angular and linear velocities, as indicated in the table of FIG. 2. At various times, the tool might be in a specific state while infinite combinations of force, torque angular and linear velocities may be used. Data reduction is achieved by using a clustering analysis in a search for a discrete number of high concentration cluster centers in the database for each one of the 15 states. The continuous 13-dimensional vectors are transformed into one-dimensional vectors of 150 symbols (10 symbols for each state that was determined by the error distortion criterion).

Data reduction can be performed in three phases. During the first phase a subset of the database is created by appending the 13-dimensional vectors associated with each state measured by the left and the right tools and performed by all subjects. The 13-dimensional subset of the database (ωₓ, ω_{y}, ω_{z}, ω_{g}, V_{Z}, Fₓ, F_{y}, F_{z}, Tₓ, T_{y}, T_{z}, F_{g}, U) was transformed into a 9-dimensional vector X̅ᵢ=[ω_{xy}, ω_{z}, ω_{g}, V_{Z}, F_{xy}, F_{xy}, F_{z}, T_{xy}, T_{z}, F_{g}] by calculating the magnitude of the angular velocity, the forces and the torques in the X-Y plane (ω_{xy}=\sqrt{ωₓ²+ω_{y}²}, F_{xy}=\sqrt{Fₓ²+F_{y}²}, T_{xy}=\sqrt{Tₓ²+T_{y}²}). This process cancels out differences between surgeons due to variations in position relative to the animal and allowed the use of the same clusters for the left and the right tools. Note the tenth dimension U was omitted. This variable is used to differentiate the Idle state (State 1) in which the tool tip is not in contact with the tissue or other elements in the scene out of all the other states (states 2-15).

The subscripts x, y and z are used to associate the angular and linear velocities (ω, V) the forces (F), and torques (T) with the stationary coordinate system and an origin located at the surgical port. The combined axes x-y, x-z and y-z define planes parallel to the coronal, sagittal, transverse planes respectively. The Z-axis is pointing toward the anterior side of the abdominal wall. The subscript g is used to associate the angular velocities (ω) and the forces (F) with the tool's grasping handle. The binary variable U indicates whether the tool is in contact with the tissue or any other element in the surgical scene.

In the second phase, a K-means vector quantization algorithm is used to identify 10 cluster centers associated with each state.

Mathematically the process is defined as follows: Given M patterns X̅₁, X̅₂, . . . , X̅_{M} contained in the pattern space S̅, the process of clustering can be formally stated as seeking the regions S̅₁, S̅₂, . . . , S̅ₖ such that every data vector X̅ᵢ (i=1, 2, . . . , M) falls into one of these regions and no X̅ᵢ is associated with two regions, i.e.
S̅₁∪ S̅₂∪ S̅₃ . . . ∪ S̅ₖ= S̅ (a) (Equation 1)
S̅ᵢ∩Sⱼ=0 ∀i≠j (b)

The K-means algorithm is based on minimization of the sum of squared distances from all points in a cluster domain to the cluster center,

$$min {\sum }_{X ∈ {S}_{i} ( k )} {( \overline{X} - {\overline{Z}}_{j} )}^{2} \qquad ( Equation 2 )$$

where Sᵢ(k) was the cluster domain for cluster center Z̅ⱼ at the k^{th} iteration, and X̅ was a point in the cluster domain.

The cluster regions S̅ᵢ represented by the cluster centers Z̅ⱼ, defined typical signatures or codeword associated with a specific state (e.g. PS, PL, GR etc.). The number of clusters identified in each type of state is based upon the squared error distortion criterion (Equation 3). As the number of clusters increased, the distortion decreased exponentially. Following this behavior, the number of clusters is increased until the squared error distortion gradient, as a function of k, decreased below a threshold of 1% that results in at least 10 cluster centers for 14 out of the 15 states. Selecting the most frequent 10 clusters for each state guarantees that the squared error distortion gradient is 1% or smaller.

$$d ( \overline{X} , \overline{Z} ) = {‖ \overline{X} - {\overline{Z}}_{j} ‖}^{2} = {\sum }_{i = 1}^{k} {( \overline{X} - {\overline{Z}}_{i} )}^{2} \qquad ( Equation 3 )$$

In a third phase, the 10 cluster centers Z̅ⱼ for each state forming a codebook of 150 discrete symbols were used to encode the entire database of the actual surgical tasks converting the continuous multi-dimensional data into a one-dimensional vector of finite symbols. This step of the data analysis facilitated the use of the discrete version of the Markov model.

FIG. 5 illustrates 10 cluster centers associated with a particular tool/tissue interaction (grasping-pushing-sweeping) in MIS as part of a codebook including 150 cluster centers representing a database of 5.5 millions data points. In grasping-pushing-sweeping, which is a superposition of three actions, the surgeon grasps a tissue or an object which is identified by the positive grasping force (F_{g}) acting on the tool's jaws and the negative angular velocity of the handle (ω_{g}) indicating that the handle is being closed. The grasped tissue or object is pushed into the port indicated by positive value of the force (F_{z}) acting along the long shaft of the tool and negative linear velocity (V_{z}) representing the fact the tool is moved into the port. Simultaneously, sweeping the tissue to the side manifested by the force and the torque in the XY plane (F_{xy}, T_{xy}) that are generated due to the deflection of the abdominal wall, the lateral force applied on the tool by the tissue or object being swept along with the lateral angular velocity (ω_{xy}) indicating the rotation of the tool around the pivot point inside the port.

Ten signatures of forces, torques, linear and angular velocities are associated with the 15 types of states (tool/tissue or tool/object interaction) defined by the table illustrated in FIG. 2. Each one of the 10 polar lines represent one cluster. The clusters were normalized to a range of [−1, 1] using the following min/max values: ω_{xy}=0.593[r/s], ω_{Z}=2.310[r/s], Vᵣ=0.059[m/s], ω_{g}=0.532[r/s], F_{xy}=5.069[N], F_{Z}=152.536[N], F_{g}=33.669[N], T_{xy}=9.792[Nm], T_{Z}=0.017[Nm].

In the graph of FIG. 5, each of the 10 polar lines represents one cluster. Each of the 15 other states or tool tissue/interactions defined in FIG. 2 is associated with 10 different and unique signatures defining a codebook with 150 symbols that can represent 5.5 million data points.

Both static, quasi-static and dynamic tool/tissue or tool/object interactions are represented by the various cluster centers. Even in static conditions, the forces and torques provide a unique and un-ambivalent signature that can be associated with each one of the 15 states.

Markov Model

In one example, data analysis included developing a model that represents the process of performing MIS and methodology for objectively evaluating surgical skill. A Markov model provides a statistical method to summarize a relatively complex task such as a step or a task of a MIS procedure. In one example, skill level was incorporated into the Markov model by developing different models based on data acquired for different levels of expertise ranging from a first year resident to an expert surgeon.

A model is generated to represent the clinical procedure for analyzing the data. The model includes multiple interconnected states where each state represents an interaction between the physician using a tool or between the physician's hands and the tissues. After the physician is engaged in a specific interaction with the tissue, different forces, torques (along with the tool kinematics) are generated through the interaction. The action/reaction information transmitted between the tool or the hand and the tissue is referred to as an observation and can be measured by an array of sensors incorporated into the various modalities previously noted.

The medical procedure can be described as a dynamic process in which the physician is moving between states while interacting with the tissue. During the physician's interaction with the tissue in each state, different types of information is exchanged between the tools or the hand and the tissue by utilizing the various observations typical to a specific state. After the physician is engaged with the tissue, the physician may remain in this state for a period of time and then perform a transition and engage with the tissue (again utilizing a different state), while using its associated observations.

This process can be modeled by a finite state machine or in a generalized form as a Markov model. The statistical nature of the model arises from the fact that each transition between two states or utilization of an observation in a state is associated with a probability. There is a particular probability that the physician will use certain transitions between the states that facilitates a specific observation while interacting in the tissue in a certain state. The model, as a whole, along with its states and observations, represents the clinical procedure. Moreover a specific navigation pattern between the model states and utilizing specific observations is associated with a particular skill. Physicians with a similar skill level are more likely to navigate through similar states of the model and leave the same trace. However, differences between the various skills level are related to different traces in the model. Each trace can be quantified by accumulating the probabilities associated with each transition. These accumulating probabilities define an objective score which can be used to differentiate between various skill levels.

The Markov model has a generic architecture (including the prime elements) such as states and observation. A specific model architecture defined for a particular medical procedure is based on an expert knowledge. Using expert knowledge, the various states and their interconnection are defined, and form a step in the model development. Each procedure has a unique model architecture and the generic methodology for assessing skill is independent of a specific procedure. The following sections will use MIS as an example of the methodology, thus demonstrating how the Markov model is translated into practice.

Analyzing the degrees of freedom (DOF) of a tool in MIS reveals that, due to the introduction of the port through which the surgeon inserts tools into the body cavity, two DOF of the tool are restricted. The six DOF of a typical open surgical tool is reduced to four DOF in a minimally invasive setup. These four DOF include rotation along the three orthogonal axes (x, y and z) and translation along the long axis of the tool's shaft (z). A fifth DOF is defined as the tool-tip jaws angle, which is mechanically linked to the tool's handle such as, when a grasper or a scissor is used. Additional one or two degrees of freedom can be obtained by adding a wrist joint to the MIS tool. The wrist joint enhances the dexterity of the tool within the body cavity.

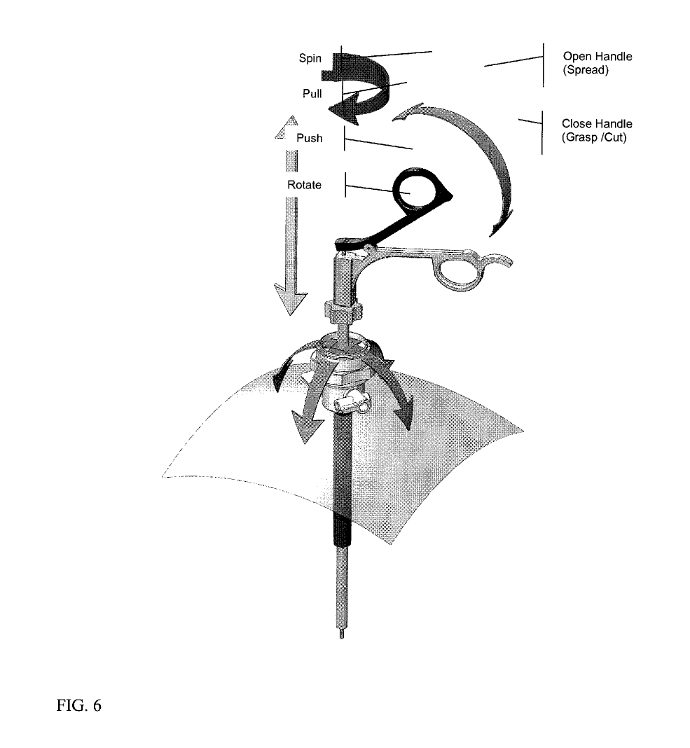

FIG. 6 illustrates five degrees of freedom in the context of a typical MIS endoscopic tool. Note that two DOF were separated into two distinct actions (Open/Close handle and Pull/Push), and the other two are combined into one action (Rotate) for representing the tool tip tissue interactions (omitted in the illustration). The terminology associated with the various DOF corresponds with the model state definitions noted in FIG. 2.

Surgeons, while performing MIS procedures, utilize various combinations of the DOF while manipulating the tool during the interaction with the tissues or other items in the surgical scene (such as a needle, a suture or a staple) in order to achieve the desired outcome. In one example, quantitative analysis of the position and orientation of the tool during surgical procedures revealed 15 different combinations of the five DOF for a tool while interacting with the tissues and other objects. These 15 DOF combinations will be further referred to, and modeled as states (see FIG. 2). The 15 states can be grouped into three types, based on the number of movements or DOF utilized simultaneously. The first type are fundamental maneuvers. The ‘idle’ state was defined as moving the tool in space (body cavity) without touching any internal organ, tissue or other item in the scene. The forces and torques developed in this state represent the interaction with the port and the abdominal wall, in addition to the gravitational and inertial forces. In the ‘grasping’ and ‘spreading’ states, compression and tension were applied on the tissue through the tool tip by closing and opening the grasper's handle, respectively. In the ‘pushing’ state, the tissue was compressed by moving the tool along the Z-axis. ‘Sweeping’ consisted of placing the tool in one position while rotating it around the X- and/or Y-axes or in any combination of these two axes (port frame). State 15 was observed in tasks involving suturing when the surgeon grasps the needle and rotating it around the shaft's long axis to insert it into the tissue. Such a rotation was not observed whenever tissue interaction was involved. With the exception of state 15, the rest of the tool/tissue interactions in Types II and III were combinations of the fundamental ones defined as Type I.

The modeling approach underling the methodology for decomposing and statistically representing a surgical task is based on a fully connected, symmetric finite-states (30 states) Markov model where the left and the right tools are represented by 15 states each as illustrated in FIG. 5. Each one of the 15 states corresponds to a fundamental tool/tissue or tool/object interaction based on tool kinematics and is associated with unique F/T and velocity signatures defined as observations and measured at the hand/tool interface and then translated to the port coordinate system of FIG. 2. In view of this model, a minimally invasive surgical task can be described as a series of finite states. In each state, the surgeon is applying a specific force/torque/velocity signature, out of 10 signatures that are associated with that state, on the tissue or on another item in the surgical scene by using the tool. The surgeon may stay within the same state for a specific time duration using different signatures associated with that state and then perform a transition to another state. The surgeon may utilize any of the 15 states by using the left and the right tools independently. The states representing the tool/tissue or tool/object interactions of the left and the night tools are mathematically and functionally linked.

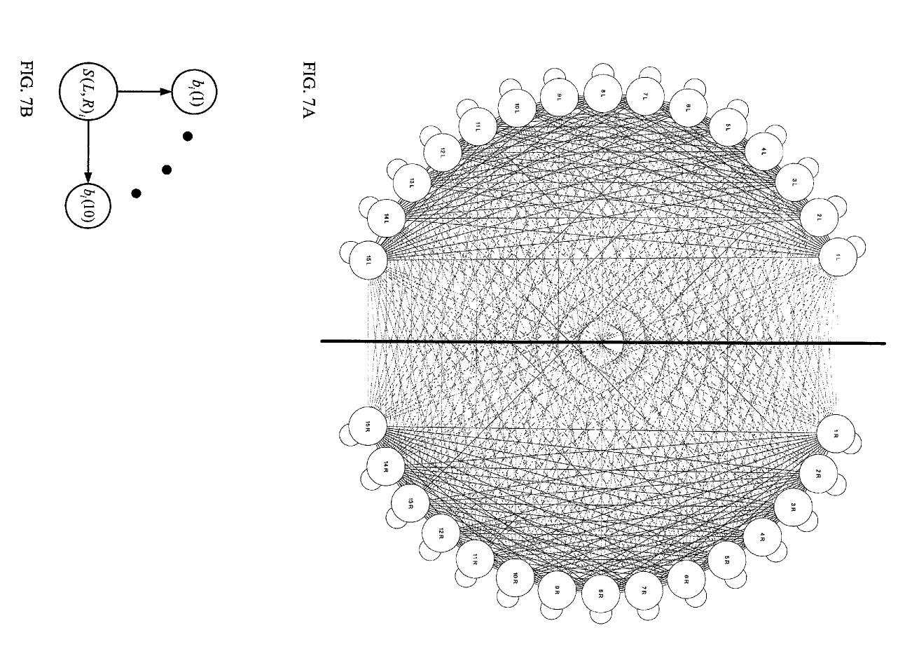

FIG. 7A illustrates a fully connected finite state diagram (FSD) for decomposing MIS. The tool/tissue and tool/object interactions of the left and the right endoscopic tools are represented by the 15 fully connected sub-models. Circles represent states whereas lines represent transitions between states. Each line that does not cross the center-line represents a probability value defined in the state transition probability distribution matrix A={aᵢⱼ}. Each line that crosses the center-line represents a probability for a specific combination of the left and the right tools and is defined by the interstate transition probability distribution matrix or the cooperation matrix C={cⱼᵣ} Note that since the probability of performing a transition from state i to state j by each one of the tools is different from probability of performing a transition from state j to state i, these two probabilities could have been represented by two parallel lines connecting state i to state j and representing the two potential transitions. For purposes of simplifying the graphical representation of A={aᵢⱼ} only one line is plotted between state i to state j.

FIG. 7B illustrates that each state out of the 15 states of the left and the right tool b(L, R), is associated with the 10 force/torque/velocity signatures or discrete observations bᵢ(1) . . . bᵢ(10) Each line that connects the state with a specific observation represents a probability value defined in the observation symbol probability distribution matrix B={bⱼ(k)}. The sub-structure associated with each state (b) is omitted to simplify the diagram.

The Markov model is defined by the notation in Equation 4. Each Markov sub-model representing the left and the right tool is defined by λ_{L} and λ_{R} (Equation 4). The sub-model is defined by:

(i) The number of states—N whereas individual states are denoted as S={s₁, s₁, . . . s_{N}}, and the state at time t as qₜ.;

(ii) The number of distinct (discrete) observation symbol—M whereas individual symbols are denoted as V={v₁, v₁, . . . v_{M}};

(iii) The state transition probability distribution matrix indicating the probability of the transition from state qₜ=sᵢ at time T to state qₜ₊₁=sⱼ at time t+1−A={aᵢⱼ}, where aᵢⱼ=P[qₜ₊₁=sⱼ|qₜ=sᵢ] 1≦i, j≦N;

Note that A={aᵢⱼ} is a non-symmetric matrix (aᵢⱼ≠aⱼᵢ) since the probability of performing a transition from state i to state j using each one of the tools is different from the probability of performing a transition from state j to state i.

(iv) The observation symbol probability distribution matrix indicating the probability of using the symbol Vₖ while staying at state sⱼ at time t−B={bⱼ(k)}, where for state j bⱼ(k)=P[vₖ at t|qᵢ=sⱼ ]1≦j≦N, 1≦k≦M;

(v) The initial state distribution vector indicating the probability of starting the process with state sᵢ at time t=1−π where πᵢ=P[q₁=sᵢ] 1≦i≦N.

The two sub-models are linked to each other by the left-right interstate transition probability matrix or the cooperation matrix indicating the probability for staying in states sᵢ with the left tool sᵣ with the right tool at time t−C={cᵢᵣ}, where cᵢᵣ=P[q_{tL}=s₁∪q_{tR}=sᵣ] 1≦l, r≦N

Note that C={cₗᵣ} is a non-symmetric matrix cₗᵣ≠cᵣₗ since it representing the combination of using two states simultaneously by the left and the right tools.

The probability of observing the state transition Q={q₁, q₂, . . . q_{T}} and the associated observation sequence O={o₁, o₂, . . . o_{T}}, given the two Markov sub-models (Equation 4) and interstate transition probability matrix, is defined by Equation 5

$$\begin{matrix}\begin{matrix}{λ}_{L} = ( {A}_{L} , {B}_{L} , {π}_{L} ) \\ {λ}_{R} = ( {A}_{R} , {B}_{R} , {π}_{R} ) \\ {a}_{ij} = \frac{n ( {q}_{t} = {s}_{j} \vert {q}_{t - 1} = {s}_{i} )}{n} \\ {b}_{jk} = \frac{m ( {v}_{k} \vert {q}_{t} = {s}_{j} )}{m ( {q}_{t} = {s}_{j} )} \\ {c}_{lr} = \frac{c ( {q}_{Lt} = {s}_{t} ⋃ {q}_{Rt} = {s}_{r} )}{n} \\ {\sum }_{j = 1}^{N} {a}_{ij} = {\sum }_{k = 1}^{M} {b}_{jk} \\ = {\sum }_{i = 1 , r = 1}^{i ∈ N , r ∈ N} {c}_{lr} = 1\end{matrix} & ( Equation 4 ) \\ P ( Q , O \vert {λ}_{L} , {λ}_{R} , C ) = {π}_{{q}_{l}} , {π}_{{q}_{ll}} {\prod }_{t = 0}^{T} {a}_{q , q , L} {b}_{q , L} ( {o}_{t} ) {a}_{{q}_{t} {q}_{t + 1} R} {b}_{{q}_{t} R} ( {o}_{t} ) {c}_{{q}_{R} {q}_{R}} & ( Equation 5 )\end{matrix}$$

Since probabilities, by definition, have numerical value in the range of 0 to 1, the probability calculated by Equation 5 converges exponentially to zero and therefore exceeds the precision range of a machine. Hence, by using logarithmic transformation, the resulting values of Equation 5 in the range of [0 1] are mapped by Equation 6 into [−∞0].

$$Log ( P ( Q , O \vert {λ}_{L} , {λ}_{K} , C ) ) = Log ( {π}_{{q}_{L}} ) + Log ( {π}_{{q}_{h}} ) + {\sum }_{i = 1}^{T} Log ( {a}_{{q}_{t} {q}_{r + t} L} ) + Log ( {b}_{q , L} ( o ) ) + Log ( {a}_{{q}_{t} {a}_{t + l} R} ) + Log ( {b}_{q , R} ( {o}_{t} ) ) + Log ( {c}_{{q}_{t} {L}_{{q}_{t}} R} ) \qquad ( Equation 6 )$$

Due to the nature of the process associated with surgery in which the procedure, by definition, always starts in the idle state (state 1), the initial state distribution vector is defined as follows in Equation 7.
π_{1L}=π_{1R}=1
λ_{iL}=πiR=0 2≦i≦N. (Equation 7)

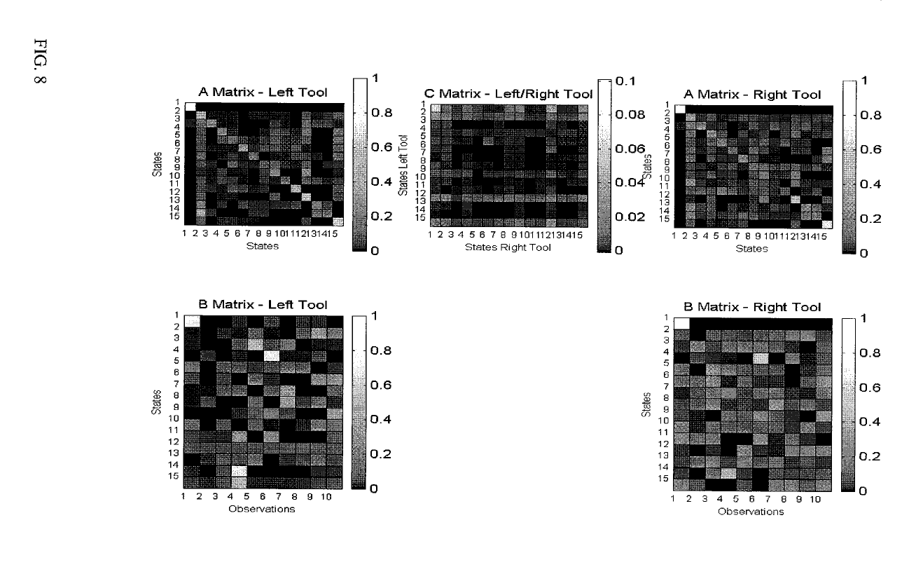

Given the encoded data, 30 Markov models, (one for each subject) are calculated defining the probabilities for performing certain tool transitions ([A] matrix), the probability of combining two states ([C] matrix), and the probability of using the various signatures in each state ([B] matrix). FIG. 8 illustrates an exemplary Markov model where the matrices [A], [B], [C], are represented as coded probabilistic maps.

An element in the [A] matrix is calculated as the ratio between the number of times a specific transition between state i to state j took place n(qₜ=sⱼ|qₜ₋₁=sᵢ) and the total number of state transitions n which is also equal to one minus the number of data points. There are N numbers of potential transition between two state and therefore the order of [A] is N×N. The sum of each line in the [A] matrix is equal to one. An element in the [B] matrix is calculated as the ratio between the number of times a specific observation vₖ was used while staying in state sⱼ, m(vₖ|qₜ=sⱼ) and the total number of visits of state j, m(qₜ=sⱼ) which is also equal to the number of times any observation was used while visiting that state. There are N number states and M number of potential transition between two states and therefore the order of [A] is N×N. The sum of each line in the [B] matrix is equal to one. An element in the [C] matrix is calculated as the ratio between the number of times the left hand side model is in state sₗ as well as the right hand side of the model is in state sᵣ, c(q_{Lt}=sₗ∩q_{Rt}=Sᵣ) and the total number of state combinations observed n which is also equal to the number of data points. The sum of all lines and columns of the [C] matrix is equal to one.

In models extracted as described above from the sample surgical data, the highest probability values in the [A] matrix appear along the diagonal. Accordingly, a transition associated with remaining at the same state is more likely to occur rather than a transition to any one of the other 15 potential states. In minimally invasive surgical suturing, for example, the default transition from any state is to the grasping state (state number 2) as indicated by the high probability values along the second column of the [A] matrix. The probability of using one out of the 150 cluster centers (illustrated in FIG. 5) is graphically represented by the [B] matrix. Each line of the [B] matrix is associated with one of the 10 states. The clusters were ranked according to the mechanical power. The left and the right tool used different distribution of the clusters. With the left tool, the most frequent clusters that were used are related to mid-range power and with the right tool, the cluster usage is more evenly distributed among the different power levels. The collaboration matrix [C] indicates that the most frequently used state with both the left and the right tools are idle (state 1), grasping (state 2) grasping pulling and sweeping (state 12). In addition, grasping rotating (state 15) with the left tool was also frequently used. Once one of the tools utilizes one of these states, the probability of using any of the states by the other tool is equally distributed between the states which is indicated by the bright stripe in the graphical representation of the [C] matrix.

Each tool (left and right) can be only in one out of the 15 states. However, there are potentially 225 (15×15) different combinations in which the left tool is in state i and the right tool is in state j. For that reason the dimensions of the [C] matrix is 15×15.

The idle state (state 1) in which no tool/tissue interaction is performed was mainly used, in most of the surgical tasks (by both expert and novice surgeons), to move from one operative state to another. The expert surgeons used the idle state as a transition state while the novices spent a significant amount of time in this state planning the next tool/tissue or tool/object interaction. In the case of surgical suturing and knot tying, the grasping state (state 2) dominated the transition phases since the grasping state, in this case, maintains the scene in an operative state in which both the suture and the needle were held by the two surgical tools.

Objective Skill Assessment

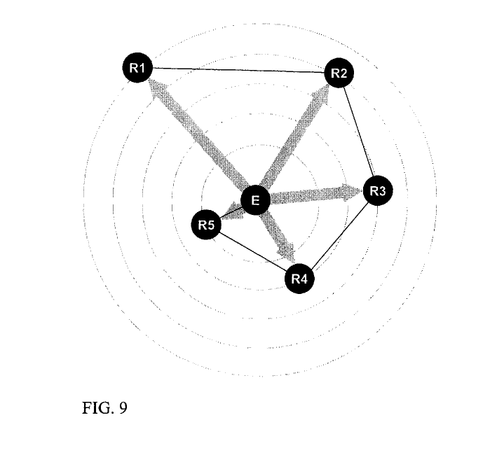

Once the Markov modes are defined for specific subjects with specific skill levels, it becomes possible to calculate the statistical distance factors between them. The statistical distance factors are considered to be an objective criterion for evaluating skill level if, for example, the statistical distance factor between a trainee (indicated by index R) and an expert (indicated by index E) is being calculated. FIG. 9 illustrates a schematic representation of the statistical distance between and expert (E) and residents (R1 . . . R5) as represented by the arrows. The statistical similarity is changing as a function of training time (moving clockwise about the expert) along as the subject's performance become similar to the experts' performance. The statistical distance indicates similarity as to the performance of the two subjects under study.

Given two Markov models λ_{Ei}=(λ_{LEi}, λ_{REi}, C_{Ei})(expert) and λ_{Ti}=(λ_{LTj}, λ_{RTj}, C_{Tj}) (trainee) the asymmetric statistical distances between them are defined as D₁(λ_{Tj}, λ_{Ei}) and D₂(λ_{Ei}, λ_{Tj}). The natural expression of the symmetric statistical distance version D_{EiTj} is defined by Equation 8.

$$\begin{matrix}{D}_{EiTj} = \frac{\begin{matrix}{D}_{1} ( {O}_{E i} , {Q}_{Ei} , {Q}_{Ti} , {Q}_{Tj} , {λ}_{E i} ) + \\ {D}_{2} ( {O}_{E i} , {Q}_{E i} , {Q}_{Tj} , {Q}_{Tj} , {λ}_{Tj} )\end{matrix}}{2} \\ = \frac{1}{2} ( \frac{log P ( {O}_{{T}_{j}} , {Q}_{{T}_{j}} \vert {λ}_{E i} )}{log P ( {O}_{Ei} , {Q}_{Ei} \vert {λ}_{E i} )} + \frac{log P ( {O}_{{T}_{j}} , {Q}_{{T}_{j}} \vert {λ}_{{T}_{j}} )}{log P ( {O}_{E i} , {Q}_{Ei} \vert {λ}_{{T}_{j}} )} )\end{matrix} \qquad ( Equation 8 )$$

Setting an expert level as the reference level of performance, the symmetric statistical distance of a model representing a given subject from a given expert (D_{EiTj}) is normalized with respect to the average distance between the models representing all the experts associated with the expert group ( D̅_{EE}) Equation 9. The normalized distance ∥D_{EiTj}∥ represents how far (statistically) is the performance of a subject, given his or her model, from the performance of the average expert.

$$‖ {D}_{{EiT}_{j}} ‖ = \frac{{D}_{{EiT}_{j}}}{{D}_{EE}} = \frac{{D}_{{EiT}_{j}}}{\frac{1}{l} {\sum }_{u = 1 ; y = 1}^{n = 5 ; y = 5} {D}_{{E}_{u} {E}_{v}}} for u \neq v \qquad ( Equation 9 )$$

For the purpose of calculating the normalized learning curve, the distances between all the subjects associated with the group of experts was first calculated D_{E}_{_{w}}_{E}_{ᵥ}−(for five subjects in the expert group—u=v=1 . . . 5-l=20) using Equation 8. The denominator of Equation 9 was then calculated.

Once the reference level of expertise was determined, the statistical distances between each one of the 25 subjects, grouped into five levels of training (R1, R2, R3, R4, R5), and each one of the experts was calculated (5 distances for each individual, 25 distances for each group of skill level and 125 distances for the entire data base) using Equation 8. The average statistical distance and its variance defines the learning curve of a particular task.

Complimentary Objective Indexes

In addition to the Markov models and the statistical similarity analysis, two other objective indexes of performance can be measured and calculated, including the task completion time and the overall length (L) of the path generated by the left and the right tool tips. Where D_{L},D_{R} are the distances between two consecutive tool tip positions P_{L}(t−1), P_{R}(t−1) and P_{L}(t), P_{R}(t) as a function of time of the left and the right tools respectively.

$$L = {\sum }_{t = 1}^{T} {D}_{L} ( {P}_{L} ( t - 1 ) , {P}_{L} ( t ) ) + {D}_{R} ( {P}_{R} ( t - 1 ) , {P}_{R} ( t ) ) \qquad ( Equation 10 )$$

These complimentary performance indexes are available for the particular surgical robot database in which motion of the tool was acquired. Acquisition of tool motion in the other modalities is also contemplated.

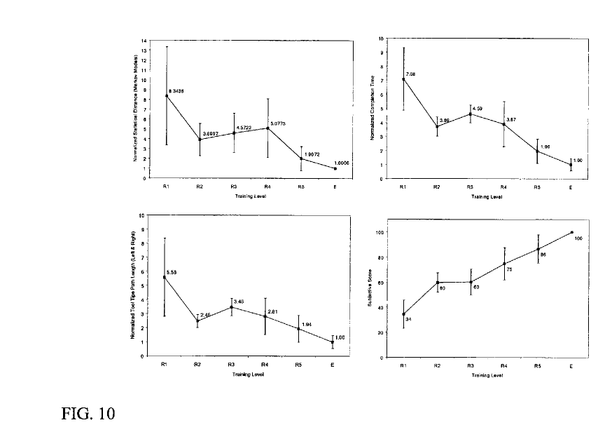

FIGS. 10A-C illustrate normalized Markov model-based statistical distance as a function of the training level, normalized completion time and normalized path length of the two tool tips respectively. The complementary subjective normalized scoring is depicted in FIG. 10D.

In particular, FIG. 10 illustrates objective and subjective assessment indexes of minimally invasive suturing learning cure. The objective performance indexes are based on: (a) Markov model normalized statistical distance, (b) normalized completion time, and (c) normalized path length of the two tool tips. In the example illustrated, the average task completion time of the expert group is 98 seconds and the total path length of the two tools is 3.832 m. The subjective performance index is based on subjective scoring of the tasks' videos and normalizing the score with respect to experts' performance (d).

The data illustrates that substantial suturing skills are acquired during the first year of the residency training. The learning curves do not indicate significant improvement during the second and the third years of training. The rapid improvement of the first year is followed by lower gradient of the learning curve as the trainees progress toward the expert level. The Markov model-based statistical distance along with the completion time criteria indicate another gradient in the learning curve that occurs during the fourth year of the residency training followed by slow conversion to expert performance. Similar trends in the learning curve are also demonstrated by the subjective assessment. One particular subject in the R2 group outperformed his peers in his own group and some subjects in a more advanced groups (R3, R4) which slightly altered the overall trend of the learning curves as defined by the different criteria.

Exemplary Method

a) acquire raw performance data.
b) use the K-means algorithm (software) to identify clusters in the database;
c) encode the entire databases using the clusters identified in (b);
d) define a Markov model for each subject performing a specific task;
e) calculate the statistical distances between the Markov models representing subjects with various skill levels and correlate these measurements with the known skill levels while defining the learning curves; and
f) to optionally validate the method of steps (a-e), perform the complimentary analysis (time, path length subjective assessment) and correlate the results with the Markov analysis (objective assessment).
   Application

A clinical procedure, regardless of the performance modality, entails synthesis between visual and kinesthetic information. Analyzing the procedure in terms of these two sources of information facilitates development of objective criteria for training physicians and evaluating the performance in different modalities including real procedures, master/slave robotic systems or virtual reality or physical simulators.

The Markov model and the vector quantization described herein is suitable for multi-modal sources of information, including low level data (such as tool kinematics and dynamics defining the model observations) and high level methodological processes (such as tool/tissue interactions formulating the model's state). The Markov model provides a mathematical representation of the process associated with manipulative tasks including complex medical procedures such as surgery. In one example, the present subject matter provides a quantitative and objective measure of surgical performance.

Exemplary outcomes of analysis of minimally invasive surgical procedures using the present subject matter revealed differences between surgeons at different skill levels including, (i) the types of tool/tissue/object interactions being used, (ii) the transitions between tool/tissue/object interactions being applied by each hand, (iii) time spent while performing each tool/tissue/object interaction, (iv) the overall completion time, (v) the various F/T/velocity magnitudes being applied by the subjects through the endoscopic tools, and (vi) two-handed collaboration. In addition, the F/T associated with each state revealed that the F/T magnitudes are relatively task-dependent with relatively high F/T magnitudes applied by novices compared to experts during tissue manipulation, and vice versa during tissue dissection. High efficiency of surgical performance was demonstrated by the expert surgeons and expressed by shorter tool tip displacements, shorter periods of time spent in the ‘idle’ state and sufficient application of F/T on the tissue to safely accomplish the task.

In various examples, the present subject matter facilitates development of objective criteria for decomposing a medical procedure and analysis using models. In one example, objective measures of skill and competency enables training and evaluating performance. In real-time, the present subject matter provides feedback to the trainee or as an artificial intelligent background layer which may increase performance efficiency in medicine and improve patient safety and outcome.

Indexes of Performance

Following two steps of data reduction, data that were collected by the surgical robot and were used to develop models representing MIS as a process. In data reduction, there is a compromise between decreasing the input dimensionality while retaining sufficient information to characterize and model the process under study. Utilizing the VQ algorithm the 13 dimensional stream of acquired data were quantized into 150 symbols with nine dimensions each.

The data quantization included identification of the cluster centers and encoding the database based on the identified cluster centers. Every data point meeting two criteria is then associated with one of the 150 identified cluster centers. The first criterion is to have the minimal geometrical distance to one of the cluster centers. Once the data point was associated with a specific cluster center it is, by definition, associated with a specific state out the 15 defined. Based on expert knowledge of surgery, the table in FIG. 2 defines the 15 states and unique sets of individual vector components. The second criterion is that, given the candidate state and the data vector, the direction of each component in the vector must match the one defined by the table for the selected state. It was indicated during the data processing that these two criteria were typically met suggesting that the data quantization process is very robust in it nature. Following the encoding process a 2-dimensional input (one dimension for each tool) was utilized to form a 30 state fully connected Markov model. The coded data with their close association to the measured data, as well as the Markov model using these codes as its observations distributed among its states, retain sufficient multi-model information in a compact mathematical formulation for modeling the process of surgery at different levels.

MIS is recognized both qualitatively and quantitatively as a multidimensional process. As such, studying one parameter (e.g. completion time, tool-tip paths, or force/torque magnitudes) reveals only one aspect of the process. A model that describes MIS as a process can facilitate study of the internal process and provide information. At the high level, a tremendous amount of information is encapsulated into a single objective indicator of surgical skill level and expressed as the statistical distance between the surgical performance of a particular subject under study from a surgical performance of an expert. As part of an alternative approach a combined score could be calculated by studying each parameter individually (e.g. force, torque, velocity, tool path, completion time etc.), assigning a weight to each one of these parameters, which is a subjective process by itself, and combining them into a single score. The assumption underlying this approach is that a collection of aspects associated with surgery may be used to assess the overall process. However this alternative approach ignores the internal process that is more likely to be revealed by a model such as the Markov model. In addition, as opposed to analyzing individual parameters, studying the low levels of the model provides profound insight into the process of MIS in a way that allows one to offer constructive feedback for a trainee regarding performance aspects like the appropriate application of F/T, economy of motion, and two handed manipulation.

The application of F/T on the tissue has an impact on the surgical performance efficiency and outcome of surgery. Some results indicate that the F/T magnitudes are task dependent. Experts applied high F/T magnitudes on the tissues during tissue dissection as apposed to low F/T magnitudes applied on the tissues by trainees that were trying to avoid irreversible damage. An inverse relationship regarding the F/T magnitudes was observed during tissue manipulation in which high F/T magnitudes applied on the tissue by trainees exposed them to acute damage. These differences were observed in particular states (e.g. those states including grasping for tissue manipulation and states involving spreading for tissue dissection). Due to the inherent variance in the data, even multidimensional ANOVA failed to identify this phenomena once the F/T magnitudes are removed from the context of the multi state model. Given the nature of surgical task, the Markov model [B] Matrix, encompassing information regarding the frequency in which the F/T magnitudes were applied, may be used to assess whether the appropriate magnitudes F/T were applied for each particular state. Tissue damage is correlated with surgical outcome and linked to the magnitudes and the directions in which F/T were applied on the tissues. As such, tissue damage boundaries may be incorporated into the [B] matrix for each particular state. Given the surgical task, this additional information may refine the constructive feedback to the trainee and the objective assessment of the performance.

The economy of motion and the two hand collaboration may be further assessed by retrieving the information encapsulated into the [A], and [C] matrices. The amount of information incorporated into these two data structures exceeds the information provided by a single indicator (such as tool-tip path length or completion time) for the purpose of formulating constructive feedback to the trainee. Given a surgical task, utilizing the appropriate sets of states and state transitions are skill dependent. This information is encompassed in the [A] matrix indicating the states that were in use and the state transitions that were performed. Moreover, the ability to refine the time domain analysis using the multi-state Markov model indicated, as was observed in previous studies, that the ‘idle’ state is utilized as a transition state by expert surgeons whereas a significant amount of time is spent in that state by trainees.

Coordinated movements of the two tools is yet another indication of high skill leveling MIS. At a lower skill level the dominant hand is more active than the non-dominant hand as opposed to a high skill level in which the two tools are utilized equally. The collaboration [C] matrix encapsulates this information and quantifies the level of collaboration between the two tools.

The Markov model provides insight into the process of performing MIS. This information can be translated into a constructive feedback to the trainee as indicated by the three model matrices [A], [B] and [C]. Moreover, the capability of running the model in real-time and its inherent memory allows a senior surgeon supervising the surgery or an artificially intelligent expert system incorporated into a surgical robot or a simulator to provide immediate constructive feedback during the process as previously described.

Although the notations and the model architecture of the Markov model and the hidden Markov model approach are similar, there are several differences between them. The Markov model can be perceived as a white box model in which each state has a physical meaning describing a particular interaction between the tools and tissue or other objects in the surgical scene (such as sutures and needles). The hidden Markov model can be perceived as a black box model in which the states are abstract and are not related to a specific physical interaction. In the white box model, each state has a unique set of observations that characterize only the specific state. By definition, once the discrete observation is matched with a vector quantization code-word the state is also defined. States in the hidden Markov model share the same observations, however different observation distributions differentiate between them.

### ADDITIONAL EXAMPLES

Other sensors can be used to generate data for the present subject matter including, for example, sensors configured to measure position, orientation, force, torque, pressure, physiological variables and contact. In addition, other sensors, including a velocity sensor, an acceleration sensor, a pressure sensor, a visual display of a scene being analyzed, a clock, and a temperature sensor can also be used to generate data for the present subject matter.

In one example, a hybrid model is generated which represents the topology between a Markov model and a hidden Markov model. The hybrid model adds another layer of complexity to the Markov model by introducing the observation elements for each state. The hybrid model provides insight into the process by linking the states to physical and meaningful interactions. The hybrid model includes the collaboration matrix [C] in addition to the Markov model notation. The collaboration matrix [C] is not normally present in either the Markov model or the hidden Markov model. The collaboration matrix [C] links the models representing the left and right hand tools since surgery is a two-handed task.

In one example, the Markov model provides physical meaning to the process being modeled. In one example, the hidden Markov model provides a compact model topology and does not rely on expert knowledge incorporated into the model.

In one example, a method of the present subject matter includes defining the scope of the model and the fundamental elements, the state and the observation. For example, in the case of minimally invasive surgery, the surgical task is modeled by a fully connected model topology were each tool/tissue/object interaction is modeled as a state. In one example, each phenomenon is represented by a model with abstract states wherein each tool/object interaction is modeled by an entire model using more generalized definitions for these interactions e.g. place position, insert remove. In one example, additional models are used with a predetermined overall structure that represents the overall process.

In one example, the scope of the model is limited to objectively assess technical factors of surgical ability. Cognitive factors can be assessed by the model where a specific action is taken as a result of a decision making process.

Decomposing MIS and analyzing it using a Markov model is one approach for developing objective criteria for surgical performance.

In one example, the present subject matter, when used in real-time during the course of learning as feedback to the trainee surgeons or as an artificial intelligent background layer, may increase performance efficiency in MIS and improve patient safety and outcome.

One example of the present subject matter utilizes a plurality of models and a performance of a specimen is correlated to a particular model based on a generated distance that describes the probability that the specimen matches a particular one of the plurality of models.

The present subject matter can be applied to other types of human machine interfaces, including, for example, flight simulators and vehicle simulators and other multi-state non-medical devices and simulators.

In one example, an intelligent layer or expert system is configured to interject a message or interrupt a process performed by a robotic device. For example, an imprudent manipulation by a low skilled surgeon will trigger delivery of a message, either visually, audibly or tactile. In one example, the robotic device will prevent an imprudent manipulation or provide cues to suggest adoption of an alternate manipulation.

In one example, the models are adapted or trained against a data set. For example, a first year resident performing a minimally invasive surgical procedure will generate a particular set of performance data. In one example, a Baum-Welch algorithm is executed by a set of computer implemented instructions. A Baum-Welch algorithm is used to train the models for each skill level based on data from the training groups of known skill levels. In other words, the Baum-Welch algorithm facilitates the determination that the hidden Markov model can generate data matching the particular specimen performance. The Baum-Welch algorithm is but one example of a class of algorithms known as forward-backward algorithms, machine learning algorithms or pattern recognition algorithms and other alorgithms are also contempalted for use with the present subject matter. In one example, a forward-backward algorithm is used to determine the probability that the specimen performance correlates to a particular Markov model.

In one example, the surgical robot is equipped with 26 sensors and at a sampling rate of 100 readings per second, 2,600 data points are generated per second.

Execution of the Baum-Welch algorithm facilitates adaptation or modification of the data to represent a particular subject performance. In one example, the Baum-Welch algorithm is executed for each particular skill level in order to train the model. In one example, specimen data is used in the forward-backward algorithm and applied to the data corresponding to each of the six models generated and the present subject matter selects the one model with the highest probability. In one example, a correlation function is executed to determine a performance grade for a particular specimen.

In one example, a “distance” is calculated between each mode and the specimen data set. The shortest distance correlates to the highest probability for a match.

In one example, a recurrent neural networks (ARMA, autoregressive moving average) is calculated to correlate specimen performance to a particular model data set.

In various examples, measurements of the tool path length (a measure of the movement of a tool tip), time, force applied or other parameter is used to judge performance. Other parameters include torque, position, displacement, electrical contact measurement (resistance) and temperature. Such parameters can be used in the analysis of surgical tasks such as suturing, cutting, cauterizing and ablating.

In one example, a hidden Markov model is applied to physical signals generated by a performance of a manipulative task conducted by a specimen. The internal parameters are adjusted to improve stability of the signal generated. For example, a window is established around a particular signal to a limit the amount of variable changes. By establishing a window or boundaries, the asymptotic change of a value is bracketed and convergence is accelerated. In one example, a trial and error approach is performed in establishing the boundaries for a particular signal value.

The present subject matter can be operated in real-time and provide feedback (any of visual, aural, tactile) regarding performance during the manipulative task.

The methodology is independent of the modality used and can be incorporated into an example of the present subject matter including any of an instrumented surgical tool, a simulator, and a robotic system. In addition, the present subject matter can include an instrumented tool configured to provide performance data where the tool is a non-surgical device.

In one example, the present subject matter executes an algorithm that can be described as a black box model of skill. The black box model generates generalized findings such as probabilities, fuzzy logic membership functions, or similar abstract numbers. In one example, the algorithm generates generalized findings of skill using a model based on fuzzy logic.

### CONCLUSION

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

What is claimed:

1. A system comprising:
a data receiver for receiving subject performance data corresponding to performance of a manipulative task by the subject;
a database including a plurality of models, each particular model in one to one relation with a particular proficiency level of a plurality of proficiency levels, wherein each model corresponds to performance of the manipulative task at a particular proficiency level; and
a processor coupled to the database and data receiver and configured to generate a specimen model corresponding to the subject performance data and configured to select a proficiency level for the subject based on proximity between the specimen model and each of the plurality of models.

2. The system of claim 1 wherein the data receiver includes at least one of a surgical robot, an instrumented tool, and a simulator.

3. The system of claim 1 further including an output device coupled to the processor.

4. The system of claim 3 wherein the output device includes at least one of a printer, a display, a transmitter, and a network interface.

5. The system of claim 1 wherein the processor is configured to execute a set of instructions for generating a model corresponding to skill.

6. The system of claim 1 wherein the processor is configured to execute a set of instructions for generating a statistical model.

7. The system of claim 1 wherein the processor is configured to execute a set of instructions for generating at least one of a Markov model and a hidden Markov model.

8. The system of claim 1 wherein the processor is configured to execute a set of instructions for generating a fuzzy logic model.

9. The system of claim 1 wherein the data receiver includes an instrumented surgical tool having an output corresponding to at least one of kinematics, contact information between the tool and a medium, and a recorded display of a surgical scene.

10. A method comprising:
receiving subject performance data corresponding to performance of a manipulative task by the subject;
accessing a database including a plurality of models, each particular model in one to one relation with a particular proficiency level of a plurality of proficiency levels, wherein each model corresponds to performance of the manipulative task at a particular proficiency level;
generating a specimen model corresponding to the subject performance data; and
selecting a proficiency level for the subject based on proximity between the specimen model and each of the plurality of models.

11. The method of claim 10 wherein generating the specimen model includes generating a model corresponding to skill.

12. The method of claim 10 wherein generating the specimen model includes generating a statistical model.

13. The method of claim 10 wherein generating the specimen model includes generating at least one of a Markov model and a hidden Markov model.

14. The method of claim 10 wherein generating the specimen model includes generating a fuzzy logic model.

15. The method of claim 10 wherein generating the specimen model includes generating model data for at least one prime element.

16. The method of claim 10 wherein selecting the proficiency level includes determining a probability.

17. The method of claim 10 wherein selecting the proficiency level includes computing a generalized finding.

18. The method of claim 10 wherein selecting the proficiency level includes computing a fuzzy logic membership function.

19. The method of claim 10 wherein receiving subject performance data includes receiving data from a plurality of sensors.

20. The method of claim 19 wherein receiving data from the plurality of sensors includes receiving data from at least one of a force sensor, a torque sensor, a position sensor, a velocity sensor, an acceleration sensor, a pressure sensor, a visual display of a scene being analyzed, a clock, and a temperature sensor.

21. A computer readable medium having instructions stored thereon for causing a computer to implement a method comprising:
receiving subject performance data corresponding to performance of a manipulative task by the subject;
accessing a database including a plurality of models, each particular model in one to one relation with a particular proficiency level of a plurality of proficiency levels, wherein each model corresponds to performance of the manipulative task at a particular proficiency level;
generating a specimen model corresponding to the subject performance data; and
selecting a proficiency level for the subject based on proximity between the specimen model and each of the plurality of models.

22. The computer readable medium of claim 21 wherein generating the specimen model includes generating a model corresponding to skill.

23. The computer readable medium of claim 21 wherein generating the specimen model includes generating a statistical model.

24. The computer readable medium of claim 21 wherein generating the specimen model includes generating at least one of a Markov model and a hidden Markov model.

25. The computer readable medium of claim 21 wherein generating the specimen model includes generating a fuzzy logic model.

26. The computer readable medium of claim 21 wherein generating the specimen model includes generating model data for at least one prime element.

27. The computer readable medium of claim 21 wherein selecting the proficiency level includes determining a probability.

28. The computer readable medium of claim 21 wherein receiving subject performance data includes receiving data from a plurality of sensors.

29. The computer readable medium of claim 28 wherein receiving data from the plurality of sensors includes receiving data from at least one of a force sensor, a torque sensor, a position sensor, a velocity sensor, an acceleration sensor, a pressure sensor, a visual display of a scene being analyzed, a clock, and a temperature sensor.

30. A system comprising:
a data receiver for receiving subject performance data corresponding to performance of a manipulative task by the subject;
at least one model corresponding to performance of the task at a particular proficiency level; and
a processor coupled to the data receiver and configured for classifying the subject performance data relative to the at least one model.

31. The system of claim 30 wherein the data receiver includes at least one of a surgical robot, an instrumented tool, and a simulator.

32. The system of claim 30 further including an output device coupled to the processor.

33. The system of claim 32 wherein the output device includes at least one of a printer, a display, a transmitter, and a network interface.

34. The system of claim 30 wherein the processor is configured to execute a set of instructions for generating at least one of a Markov model and a hidden Markov model.

35. The system of claim 30 wherein the processor is configured to execute a set of instructions for generating a model corresponding to skill.

36. The system of claim 30 wherein the processor is configured to execute a set of instructions for generating a statistical model.

37. The system of claim 30 wherein the processor is configured to execute a set of instructions for generating a fuzzy logic model.
